(19) United States
(12) Patent Application Publication
(10) Pub. No.: US 2022/0098555 A1
(43) Pub. Date: Mar. 31, 2022

(54) METHOD FOR VIRUS PRODUCTION

(71) Applicant: OLOGY BIOSERVICES, INC., ALACHUA, FL (US)

(72) Inventor: ERIC VELA, BELL, FL (US)

(21) Appl. No.: 17/310,390

(22) PCT Filed: Feb. 14, 2020
(86) PCT No.: PCT/US2020/018347

§ 371 Date: Jul. 30, 2021

Related U.S. Application Data
(60) Provisional application No. 62/806,277, filed on Feb. 15, 2019.

(51) Int. Cl. C12N 7/00; C12M 1/12; C12M 1/34

(52) U.S. Cl. CPC C12N 7/00; C12M 25/18; C12M 41/34; C12M 41/26; C12N 2760/20234; C12M 41/36; C12N 2760/20251; C12N 2760/20243; C12M 41/12

(57) ABSTRACT

The invention relates to a method of increasing the yield of virus, virus particles, or viral vectors from host cells in a fixed-bed bioreactor by specifically modifying the dissolved oxygen levels in the media.

### CROSS-REFERENCE TO RELATED APPLICATIONS

This application claims the benefit of the U.S. Provisional Application No. 62/806,277 filed on Feb. 15, 2019, which is incorporated by reference herein in its entirety.

### FIELD OF INVENTION

The present invention relates to a method of propagating virus and viral vectors for vaccine and virus vector manufacturing. More particularly, the invention relates to a specific method for increasing virus yield from host cells in a fixed-bed bioreactor.

### BACKGROUND OF THE INVENTION

Robust technologies that allow rapid production of viruses and virus vectors to meet the ever-increasing demand for vaccines and other therapeutics are essential. In addition, for the development of versatile host cell technology platforms such as Vero cells and other mammalian cell platforms, avian cell platforms, and insect cell technology platforms, technologies that improve the virus yield from the host cells also play an important part in accelerating the development of the vaccine process and production. The present invention fulfills a need to improve methods of virus generation.

### BRIEF SUMMARY OF THE INVENTION

The present invention provides a process of increasing virus yield. In one embodiment, a method for producing virus in a bioreactor is disclosed. The method comprises of the steps: 1) providing host cells in a bioreactor in an environment where parameters such as dissolved oxygen (dO₂), pH, and temperature can be controlled, 2) growing the cells at a constant initial 100% dO₂, 7.4 pH and 37° C. temperature, 3) decreasing the dO₂ level to 20-50% of the initial dO₂ level, while keeping the pH and temperature constant, 4) infecting the cell with at least one virus 8-24 hours after decreasing dO₂, incubating the host cell with the virus at dO₂ level of 20-50%, pH of 7.4 and 37° C. temperature, and 5) harvesting the virus. In an embodiment, the host cells are adherent cells that are anchorage-dependent and require microcarriers and/or a fixed-bed to anchor. In a preferred embodiment, Vero cells are used as host cells in a fixed-bed bioreactor.

In a preferred embodiment of the invention, the dO₂ is decreased at least 50%, at 12 hours before infecting the host cells with a virus. In a preferred embodiment, the host cells are infected with the virus after the host cells are grown to the highest cell density. In yet another embodiment of the invention, the dO₂ is decreased after the host cells have reached the highest cell density.

### BRIEF DESCRIPTION OF THE DRAWINGS

FIG. 1A illustrates microcarrier strips that are used in a fixed-bed bioreactor, 13 microcarrier strips of about 11.2 cm² 3-dimensional area per strip are shown in 5 mL of media.

FIG. 1B illustrates a cross-section of a bioreactor that may be used to hold up to 3,500 strips per bed.

FIG. 1C illustrates the different parts of the bioreactor in a cross-sectional view.

FIG. 2 is a graphical illustration of the dO₂, pH, temperature, and biomass parameters. The dO₂, pH, and temperature were set as constants and infection occurred when the conductivity associated with the biomass probe (which measures the increase in cell growth) reached 55 mS/cm.

FIG. 3 is a graphical illustration of the dO₂, pH, temperature, and biomass parameters. The pH and temperature were set as constants, while the dO₂ was decreased to 45 to 50% 12 hours before infection and remained constant throughout infection. At the time of infection, the conductivity associated with the biomass probe reached 80 mS/cm.

FIG. 4 is a graphical illustration of the dO₂, pH, temperature, and biomass parameters. The dO₂, pH, and temperature were set as constants. At the time of infection, the conductivity associated with the biomass probe reached 110 mS/cm.

### DETAILED DESCRIPTION OF THE INVENTION

The present invention provides methods for increasing the production of virus in a bioreactor.

The following applies to the detailed description section of this application.

Where an indefinite or definite article is used when referring to a singular noun, e.g. “a”, “an” or “the”, this includes a plural of that noun unless something else is specifically stated. In the context of the present invention, the terms “about” or “approximate” denote an interval of accuracy that the person skilled in the art will understand to still ensure the technical effect of the feature in question. The term typically indicates a deviation from the indicated numerical value of ±10%, and preferably of ±5%.

The present invention relates to a method of producing a virus in a bioreactor comprising the steps of a) growing host cells in a constant initial dO₂ level, pH, and temperature; c) decreasing the dO₂ to 20-50% of initial level, 8-24 hours before infection; d) infecting the cells with at least one virus; and e) harvesting the virus. The quantity of virus produced by this method is significantly more than that produced in a conventional method where all the parameters including dO₂ are kept constant throughout the process.

As used herein, the term “large-scale” production means the production in a minimum cultivation volume of at least 200 liters, preferably of at least 500 liters, most preferably of about 1000 liters.

As used herein, the term “bioreactor” refers to a device that supports a biologically active environment in which a biological process such as propagation of virus and vectors under controlled conditions may be carried out. Bioreactors may be designed for small-scale cultures such as those used in research laboratories, as well as large-scale bioreactors comprising vessels or vats to produce and harvest biological macromolecules such as vaccine virus, antigens, and vectors on a pilot plant or commercial scale. A bioreactor may be used to propagate both suspended and adherent cells. The bioreactor is a controlled environment wherein the oxygen/dO₂, nitrogen, carbon dioxide, and pH levels may be adjusted. Parameters such as dO₂, pH, temperature, and biomass are measured at periodic intervals. The “capacity” of the bioreactor may from range 5 mL to 5000 mL. The capacity may be about 2 mL to about 10 mL, from about 5 mL to about 50 mL, from about 25 mL to about 100 ML, from about 75 mL to about 500 mL, from about 250 mL to about 750 mL, from about 600 mL to about 1000 mL. In a preferred embodiment, the capacity may be 50 mL or 80 mL. In another preferred embodiment, the capacity may be 700 mL to 800 mL.

A “fixed-bed bioreactor” means a type of bioreactor which includes a fixed-bed of packing material that promotes cell adhesion and growth. Fixed-bed bioreactors have been used to produce viral vaccine products at both small and large-scale due to the ability to perfuse high-cell densities with low shear force. The fixed-bed bioreactor may be a single-use bioreactor such as the commercially available iCELLis system (Pall Corporation). The iCELLis system platform offers a novel fixed-bed technology comprising carriers composed of woven medical-grade polyethylene terephthalate (PET) fibers in a robust, single, closed system that does not require any aseptic handling. Additionally, this system incorporates high rates of gas exchange using “waterfall” technology through the control of temperature, O₂, pH, carbon dioxide (CO₂), and nitrogen (N₂), in addition, the use of a magnetic impeller that produces low cell shear stress and evenly distributed media circulation. For most viruses, production titers from the iCELLis system are significantly increased when compared to classical adherent cell flat-stock flasks. The iCELLis technology may be used at small-scale such as in the iCELLis Nano, where the growing area is between 0.5 to 4 m² and manufacturing scale, such as in iCELLis 500 where the growing area ranges from 66 to 500 m². Processes developed in the small-scale system may be scaled up to that of the manufacturing scale.

A fixed-bed bioreactor may have sensors that measure and monitor the pH, temperature, dissolved oxygen, and the biomass, which indicates adherent cell density. A fixed-bed bioreactor may also have different ports that enable the addition of oxygen or nitrogen, a media exchange port, ports for the addition of sodium hydroxide (NaOH) and/or CO₂ to adjust the pH. The dO₂ of the media may be modified by addition of O₂ or N₂. Preferably the dO₂ levels may be depleted in a controlled manner by injecting N₂ in the headspace of the bioreactor, simultaneously stirring and monitoring the dO₂.

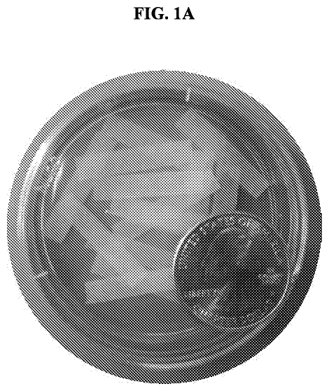

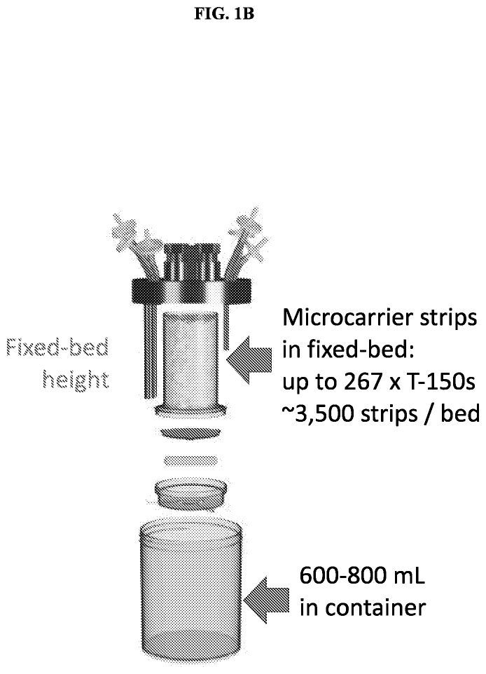

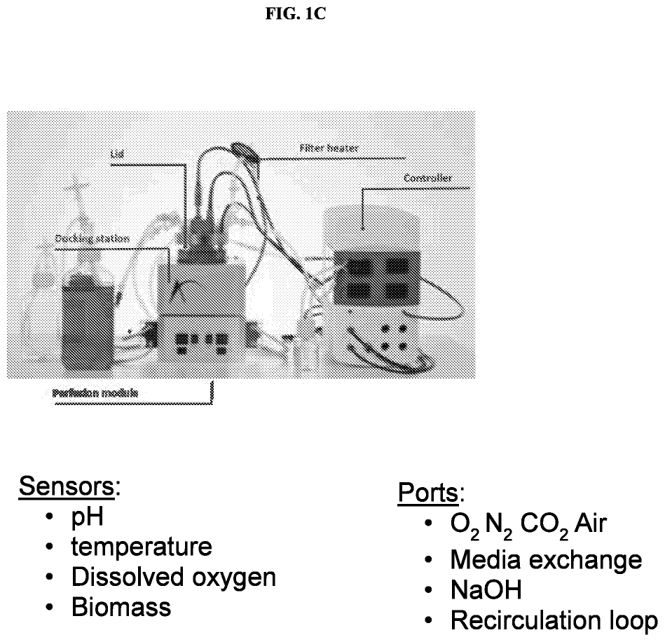

The host cell of the disclosed method may be an anchorage-dependent cell or adapted to be an anchorage-dependent cell line. The host cells of the disclosed method may be cultivated on microcarriers, which may be in suspension in bioreactors or on microcarrier strip. Preferably, the host cells are cultivated on microcarrier strips in a fixed-bed of a fixed-bed bioreactor. Each microcarrier strip may provide 1.25 cm² 2-dimensional area and 11.2 cm² 3-dimensional area per strip. About 13 microcarrier strips may provide an approximate area of 145.6 cm² which is roughly equal to the growth area provide by one T-150 flat-stock flask. Preferably, the fixed-bed bioreactor is a commercially available iCELLIS Nano (Pall Corporation), iCELLis 500 bioreactor (Pall Corporation), or a Univercells fixed-bed bioreactor (Univercells SA). The fixed-bed may provide a maximum of 40,000 cm² in an 800 mL fixed-bed bioreactor such as the iCELLis Nano, and up to 5,000,000 cm² in a 25 L fixed-bed bioreactor such as iCELLis 500 (FIG. 1A-C; Table 1). The fixed-bed height may range from 20 mm and 10 mm, providing a growth area of 5300 cm² to 40,000 cm² in an 800 mL fixed-bed bioreactor to 660000 cm² to 5,000,000 cm² in a 25 L fixed-bed bioreactor.

TABLE 1

| | iCELLis Nano: 800 mL | | | | iCELLis 500: 80 L | | | |
|---|---|---|---|---|---|---|---|---|
| | Compaction (strips per bed) | | | | | | | |
| | Low | | High | | Low | | High | |
| Total Surface Area/Equivalent T-150 | cm² | T-150 s | cm² | T-150 s | cm² | T-150 s | cm² | T-150 s |
| Fixed-bed height - 20 mm | 5,300 | 35 | 8,000 | 53 | 6.60E+05 | 4,400 | 1.00E+06 | 6,666 |
| Fixed-bed height - 40 mm | 10,600 | 70 | 16,000 | 1065 | 1.33E+06 | 8,867 | 2.00E+06 | 13,333 |
| Fixed-bed height - 100 mm | 26,000 | 173 | 40,000 | 267 | 3.03E+06 | 22,000 | 5.00E+06 | 33,333 |

Host cells may be cultivated by using a seeding density ranging from 2000 to 20,000 cells per cm². The seeding density may be adjusted based on the type of host cell, the volume of the bioreactor, the height of fixed-bed in a fixed-bed bioreactor, etc. It is within the knowledge of one skilled in the art to select the optimum seeding density for the process. The growth of cells may be measured by measuring the biomass, using a biomass sensor within the fixed-bed of the bioreactor. The biomass, which indicates the mass of the adherent cells, through conductivity, may be utilized to monitor the overall growth of host cells and the decrease in the cell mass due to the propagation of virus after infection. Higher biomass indicated by higher conductivity as monitored by the biomass sensor, indicates a higher growth rate of the cells. The biomass may range from a low conductivity of 5 mS/cm at low biomass at the beginning of cultivation to about 110±50 mS/cm at maximum biomass when the cells may have reached maximum growth.

As used herein, “culture media” or “media” refers to a liquid used to culture the host cells in the bioreactor. The media used in the procedure of the disclosure may include various ingredients that support the growth of the host cells, including but not limited to amino acids, vitamins, organic and inorganic salts, carbohydrates. The media may be serum-free media, which is media formulated without any animal serum. A serum-free media when used be selected from, but not limited to, DMEM, DMEM/F12, Medium 199, MEM, RPMI, OptiPRO SFM, VP-SFM, VP-SFM AGT, HyQ PF-Vero, MP-Vero. The culture media may also be animal-free media; that is, it does not have any product of animal origin. The culture media may also be protein-free media; that is, the media is formulated with no proteins. The serum-free or protein-free media may be formulated without serum or protein but may contain cellular protein derived from the host cells, and optionally proteins specifically added to the serum-free or the protein-free media.

The pH for cultivation can be, for example, between 6.5-7.5, depending on the pH stability of the host cells. Preferably the cells are cultivated at a pH of 7.4. The host cells may be cultivated at the temperature between 20-40° C., specifically between 30 and 40° C., and preferably at 37° C. for mammalian cells.

The host cell or host cell line or cells used for the cultivation of virus in the method of the disclosure may be any eukaryotic cell that is suitable for the production of virus antigen, viral vector, or virus production. Preferably the host cell may be “adherent cell” or an “anchorage-dependent cell.” Adherent cells are cells that adhere to a surface in culture condition, anchorage may be required for their grown, and they may also be called anchorage-dependent cells. Adherent cells suitable for the procedure of the disclosure include but not limited to Vero cells, MBCK cells, MDBK cells, MRC-5 cells, BSC-1 cells, LLC-MK cells, CV-1 cells, CHO cells, COS cells, murine cells, human cells, avian cells, insect cells, HeLa cells, HEK-293 cells, MDOK cells, CRFK cells, RAF cells, TCMK cells, LLC-PK cells, PK 15 cells, W1-38 cells, T-FLY cells, BHK cells, SP2/0 cells, NSO cells, NTCT cells, and PerC6 cells, 3T3 cells, or a combination or modification thereof. The preferred adherent cell is an anchorage-dependent cell that may be grown on a carrier such as a PET strip, but suspension cells that may be adapted to grow as adherent cells may also be used. More preferably, the anchorage-dependent cells of the disclosure are Vero cells. It is within the knowledge of one skilled in the art to select an adherent host cell suitable for use in the process of the disclosure.

The virus of the disclosure may be a virus, virus antigen, or viral vector or combination or modification thereof. The virus may be a whole virus, or a virus antigen selected from a group of but not limited to Vascular Stomatitis virus (VSV), Adenovirus, Influenza virus, Chikungunya virus, Ross River virus, Hepatitis A virus, Vaccinia virus and recombinant Vaccinia virus, Japanese Encephalitis virus, Herpes Simplex virus, Cytomegalovirus (CMV), Rabies virus, West Nile virus, Yellow Fever virus, and chimeras thereof, as well as Rhinovirus and Reovirus.

In an embodiment of the disclosure, the virus is a virus vector. Viral vectors are viruses that may be used to transfer passenger nucleic acid sequences into a cell of interest. The viral vector may be a viral expression vector that may be used to derive recombinant proteins. Preferably, the viral vector may a modified Vaccinia virus Ankara (MVA), VSV, adeno-associated virus (AAV), lentivirus, retrovirus, adenovirus. More preferably, the viral vector of the invention is the VSV vector. The recombinant protein expressed by the viral vector may be a viral protein, a bacterial protein, a therapeutic recombinant protein, or a combination thereof. More preferably, the recombinant protein produced by the viral vector is a viral protein.

Preferably, the virus of the invention is a VSV vector. VSV, a member of the family Rhabdoviridae, is an enveloped virus with a negative-stranded RNA genome that causes a self-limiting disease in live-stock. Attenuated VSV are desirable viral vectors, as they are non-pathogenic in humans, almost non-virulent in animals, show robust growth in continuous mammalian cell lines of interest, lack a DNA intermediate during replication, elicit strong cellular and humoral immune response, and a genomic structure that allows insertion of transgenes at multiple sites (Humphreys and Sebastian, Immunology, 2018, 153:1-9; Clarke et al., Vaccine. 2016 34:6597-6609).

As used herein, “infection” or “virus infection” refers to the entry of a virus into the host cell and the subsequent replication of the virus in the cell. The infection of a host cell in the method of the disclosure may be carried out when the cells reach a specific biomass. Preferably, the cells may be infected when they reach the high growth rate, indicated by high biomass, and high conductivity as measured by the biomass sensor. The cells may be infected with the virus of interest when the conductivity ranges from 50 mS/cm to about 120±20 mS/cm. The cells are preferably infected when the cells have reached a high growth shown by a conductivity of 110±10 mS/cm. The host cells are infected by at least one virus particle. As used herein, multiplicity of infection (MOI) is the average number of virus particles infecting each cell. The infection of the host cells with the virus can be carried out at an MOI of about 0.0001 to 10, preferably of 0.001 to 0.5, and most preferably at an MOI of 0.05. The number of virus particles necessary for sufficient infection is within the knowledge of one skilled in the art.

The host cells of the method of the disclosure may be cultivated at an initial dO₂ of 100%. The dO₂ may be decreased to a level of 90% to a level as low as 20%, prior to infection. The dO₂ may be decreased from about 80% to about 60%, from about 70% to about 40%, from about 50% to about 15%. Preferably, the dO₂ may be decreased from about 50% to approximately 20%, before infection. More preferably, the level is decreased to about 20% before infection.

The dO₂ may be decreased starting at a time ranging from 2 to 24 hours prior to infection and kept at this level throughout the entire infection process and through the harvest of the virus. The dO₂ is decreased starting from about 2 hours to about 10 hours, from about 5 hours to about 15 hours, from about 10 hours to about 20 hours, and from 18 hours to about 24 hours before infection. Preferably the dO₂ is decreased starting at a time ranging from 8 hours to approximately 12 hours before infection.

The decrease in dO₂ of the disclosure may be initiated when the conductivity, as measured by the biomass sensor ranges from about 50 mS/cm to about 90 mS/cm. The decrease in dO₂ of the disclosure may be initiated when the conductivity ranges from about 40 mS/cm to about 60 mS/cm, from about 50 mS/cm to about 80 mS/cm, from about 70 mS/cm to about 90 mS/cm, from about 80 mS/cm to about 100 mS/cm. Preferably, the decrease in dO₂ of the disclosure is initiated when the conductivity ranges from about 70 mS/cm to about 90 mS/cm.

“Harvesting” or “virus harvesting” as used herein refers to the collection of the virus, by collecting unclarified culture media from the host cell in the bioreactor. The harvesting of the virus may be performed 2 to 5 days post-infection, or 3 to 6 days post decrease of dO₂. Preferably harvesting of the virus may be performed 2-days post-infection. Some viruses may require an addition step of host cell lysis before harvest.

Viruses of the disclosure may be quantified by methods including but not limited to plaque assays, end-point dilution assays, hemagglutination assays, bicinchoninic acid assay, or electron microscopy. Preferably, the virus may be quantified by a plaque assay method. As used herein, a plague assay method is a method to measure the number of infectious virus particles, based on its measurement of plaque-forming units (pfu). In the plaque assay, cell monolayers are infected with a serial dilution of the virus stock solution, and an agarose overlay is used to restrict the flow of virus. The infected cells release progeny virus, which in turn infect neighboring cells. The cells are lysed to produce clear regions surrounded by uninfected cells, called plaques, which are visualized using a dye. A higher sample virus titer leads to a higher number of plaques.

### EXAMPLES

The iCELLis Nano fixed-bed bioreactor system was used in Example 1-4. The iCELLis Nano bioreactor can hold about 800 mL, which is equivalent to about 5,300 to 40,000 total surface growth area with a fixed-bed height of 20 mm to 10 mm. The growth area was equivalent to 35 to 267 T-150 flasks that could be used for stacked growth (see FIG. 1A, 1B, and Table 1). Runs with different parameters were performed with the iCELLis.

### Example 1. VSV Production from a Campaign where the Parameters Served as a Baseline for Virus Production

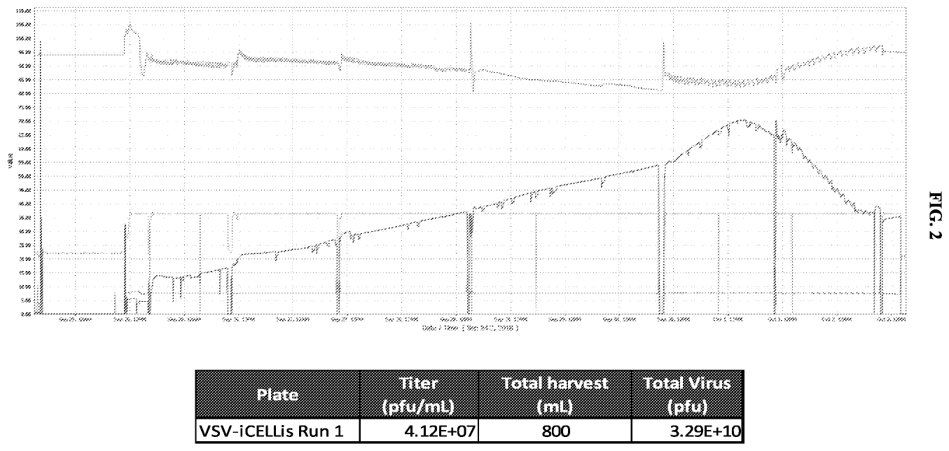

Vero cells were grown at approximately 100% dO₂, 37° C. temperature, 7.4 pH in iCELLis bioreactor. Over the cultivation period of the Vero cells, the biomass sensor of the bioreactor was used to monitor cell growth, and the Vero cells were infected with VSV at 55 mS/cm conductivity (a measure of cell growth). The system reached the highest conductivity (highest cell growth) of about 75 mS/cm about 12-24 hours after infection (FIG. 2). The infection was at 0.05 MOI. The virus was harvested 2 days post-infection. Virus production was increased in excess of 1 log per mL when compared to titers from the same cells growing in flat-stock (FIG. 2; Table 2).

### Example 2. VSV Production from a Campaign where the dO₂ was Decreased for Approximately 12 Hours Prior to Infection and Maintained Through Infection to Harvest

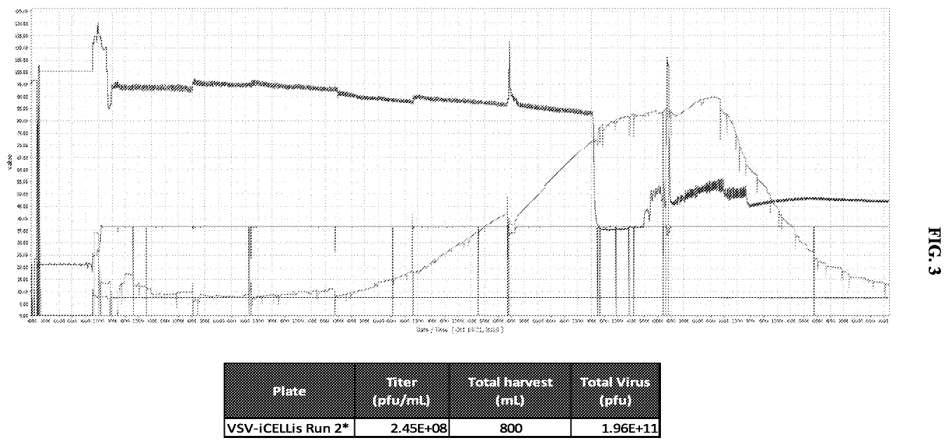

Vero cells were cultivated at approximately 100% dO₂, 37° C. temperature, 7.4 pH. Over the cultivation period of the Vero cells, the biomass sensor was used to monitor cell growth, and the cells were infected at 80 mS/cm conductivity (approximately highest conductivity), i.e., the Vero cells were infection when maximum cell growth was reached. Approximately 12 hours before infection the dO₂ level was lowered to 45-50% and kept constant at this decreased level throughout infection and through harvest. The temperature was maintained at 37° C., and pH was maintained at 7.4. The virus was harvested approximately 2 days after infection. Decreasing the dO₂ 12 hours prior to infection, infecting Vero cells VSV after the Vero cells achieved maximum cell growth, resulted in a VSV titer increase of over 2 logs when compared to VSV titer from flat-stock (Table 2) and a 5.9× increase in viral titer when compared to that of Example 1 (no dO₂ decrease) (FIG. 3).

### Example 3. VSV Production from a Campaign with Maximum Conductivity in Addition to the dO₂, pH, and Temperature Remaining Constant

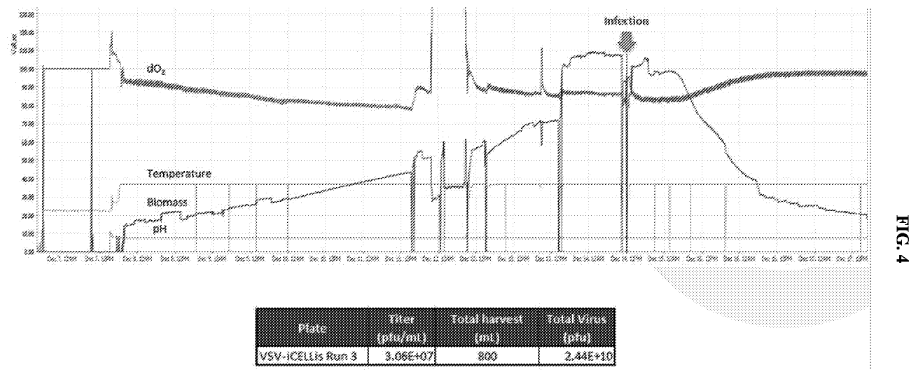

Vero cells were cultivated at approximately 100% dO₂, 37° C. temperature, 7.4 pH in the bioreactor. Over the cultivation period of the Vero cells, the biomass sensor was used to monitor cell growth, and the cells were infected at 110 mS/cm conductivity (approximately highest conductivity, and therefore when maximum cell growth was reached). The infection was at 0.05 MOI. No adjustment was made to the dO₂ levels. The temperature was maintained at 37° C., and pH was maintained at 7.4 throughout the cultivation and infection period. The virus was harvested approximately 2 days post-infection. The VSV titer from this experiment was similar to that of Example 1, showing that the higher yield observed in Example 2 was due the modification of the dO₂ and not due to infecting the Vero cells at highest cell growth, which could presumably increase the overall titer to due to more cells becoming infected (FIG. 4; Table 2).

TABLE 2
Viral Titers from Bioreactor Production

| | C510 Flatstock | Run 1 | Run 2 | Run 3 |
|---|---|---|---|---|
| O₂% during Infection | NA | 90% | 40% | 20% |
| Titer (pfu/mL) | 1.30E + 06 | 8.4 E + 06 | 9.17E + 07 | 1.13E + 08 |
| Total Virus Production (pfu) | 1.30E + 09 | 6.9E + 09 | 7.33E + 10 | 9.07E + 10 |

Table 2 compares the propagation data between different runs. The propagation data was compared between: 1. VSV propagated from Vero cells in a flat-stock flask, 2. VSV propagated from Vero cells in an iCELLis system (Run 1), where dO₂% during infection is 90%, 3. VSV propagated from Vero cells in an iCELLis system (Run 2) where dO₂% during infection is 40%, and 4. VSV propagated from Vero cells in an iCELLis system (Run 3) where dO₂% during infection is 20%. The data in Table 2 shows a significant increase in VSV titer, and total virus production progressively, from CS10 flask-stock, Run 1, Run 2, and Run 3, respectively. This shows that using the flat-bed bioreactor iCELLis to propagate VSV resulted in a 1 to 2 log increase in virus production per mL when compared to virus produced from flat-stock. More significantly, a progressive decrease in dO₂ during infection, resulted in a progressively significant increase in VSV titer and total virus production.

### Example 4. VSV Production at Different dO₂ Levels at Infection

VSV was grown in Vero cells at approximately 100% dO₂, 37° C. temperature, 7.4 pH. Approximately 12 hours before infection the dO₂ level was lowered to 90%, 40%, and 20% and kept constant at this decreased level throughout infection. The temperature was maintained at 37° C., and pH was maintained at 7.4. The virus was harvested approximately 2 days after infection. The results, as shown in Table 2, show a progressive increase in virus yield with the level of dO₂ decrease at infection.

I claim:

1. A method of producing virus in a bioreactor comprising the following steps:
a) providing host cells in the bioreactor;
b) growing host cells in a constant initial dO₂ level, pH, and temperature;
c) decreasing the dO₂ to 20-90% of initial oxygen level;
d) infecting the host cells with at least one virus or virus particle 2-24 hours after step c);
e) incubating said host cells infected with said virus or virus particle to propagate said virus; and
f) harvesting the virus.

2. The method of claim 1, wherein the host cells are adherent cells.

3. The method of claim 1, wherein the bioreactor is a flat-bed bioreactor.

4. The method of claim 1, wherein the height of the fixed-bed is 2 cm to 10 cm.

5. The method of claim 1, wherein the bioreactor is a single-use flat-bed bioreactor.

6. The method of claim 1, wherein said dO₂ level in step c is decreased to 20-50%.

7. The method of claim 1, wherein said dO₂ level in step c is decreased when the conductivity is 60 mS/cm to 80 mS/cm.

8. The method of claim 1, wherein the host cell in step d is infected with a virus when the conductivity is 90 mS/cm to 110 mS/cm.

9. The method of claim 1, wherein the infection of the host cells with the virus is at multiplicity of infection (MOI) of about 0.1 to 0.05.

10. The method of claim 1, wherein, the infection of the host cells with the virus is at MOI of 0.05.

11. The method of claim 1, wherein the dO₂ in step c is decreased 12 to 24 hours prior to infecting the host cell in step d.

12. The method of claim 1, wherein the virus is selected from a group consisting of VSV, adenovirus, Influenza virus, Ross River virus, Hepatitis A virus, Vaccinia virus and recombinant Vaccinia virus, Herpes Simplex virus, Japanese Encephalitis virus, Herpes Simplex virus, West Nile virus, Yellow Fever virus, and chimeras thereof, as well as Rhino virus and Reovirus.

13. The method of claim 1, wherein the virus is a viral vector.

14. (canceled)

15. The method of claim 1, wherein the host cells are selected from the group consisting of Vero cells, MBCK cells, MDBK cells, MRC-5 cells, BSC-1 cells, LLC-MK cells, CV-1 cells, CHO cells, COS cells, murine cells, human cells, avian cells, insect cells, HeLa cells, 293 cells, MDOK cells, CRFK cells, RAF cells, TCMK cells, LLC-PK cells, PK 15 cells, W1-38 cells, T-FLY cells, BHK cells, SP2/0 cells, NSO cells, and PerC6 cells.

16. (canceled)

17. The method of claim 1, further comprising the step of determining the virus titer by plaque assay method.

18. The method of claim 1, further comprising the step of purifying and or characterizing of the virus.

19. The method of claim 1, further comprising the step of producing a vaccine with said virus.

20. The method of claim 1, wherein the bioreactor has a capacity of 700 to 800 mL or has a capacity of 50 to 80 L.

21. (canceled)

22. The method of claim 1, wherein the bioreactor includes protein-free media.

23. A method of producing virus in a bioreactor comprising the following steps:
a) providing host cells in the bioreactor;
b) growing host cells in a constant initial dO₂ level, pH, and temperature to confluence;
c) decreasing the dO₂ to 20-50% of initial dO₂ level;
d) infecting the host cells with at least one virus or virus particle;
e) incubating said host cells infected with said virus or virus particle to propagate said virus; and
f) harvesting the virus.
